(19) United States
(12) Patent Application Publication
(10) Pub. No.: US 2025/0339048 A1
(43) Pub. Date: Nov. 6, 2025

(54) Continuous Real-Time Monitoring of Hydrocephalus Shunt Function

(71) Applicant: Jonathan Socha, Baltimore, MD (US)

(72) Inventor: Jonathan Socha, Baltimore, MD (US)

(21) Appl. No.: 19/200,411

(22) Filed: May 6, 2025

Related U.S. Application Data
(60) Provisional application No. 63/643,032, filed on May 6, 2024.

(51) Int. Cl. A61B 5/03; A61B 5/00

(52) U.S. Cl. CPC A61B 5/031; A61B 5/0031; A61B 5/6852; A61B 5/742; A61B 5/746; A61B 2560/0219; A61B 2562/0247; A61B 2562/04

(57) ABSTRACT

A system for monitoring cerebrospinal fluid (CSF) intracranial pressure and shunt flow in a hydrocephalus patient. It may include a ventricular catheter, a peritoneal catheter and a valve in fluid communication with the catheters. Piezoelectric pressure sensors may be disposed on each end of the catheters. An intracranial electret pressure sensor may be disposed on the ventricular cavity end of the ventricular catheter. A processing and transmission module may determine pressure values at each of said sensors, compute shunt flow comprising ventricular catheter and peritoneal catheter flow rates, and cause a wireless data transmitter to transmit a CSF pressure value and shunt flow to an external receiver.

### CROSS-REFERENCES TO RELATED APPLICATIONS

This application claims the benefit of U.S. Provisional Application Ser. No. 63/643,032 filed May 6, 2024, the contents which are hereby incorporated by reference.

### BACKGROUND OF INVENTION

### Field of the Invention

The present invention relates generally to medical instruments and more specifically it relates to early detection of hydrocephalus shunt malfunction and measurement of intracranial pressure of patient hydrocephalus.

### Description of Related Art

To diagnose a shunt malfunction, a hydrocephalus patient often undergoes invasive testing in the clinic. The physician inserts a needle into the shunt reservoir and attempts to draw Cerebral Spinal Fluid (CSF). If this proves unsuccessful, the surgeon must make an incision to reach the shunt valve. She/he then disconnects the catheter from the valve and depending on the flow of CSF, determines where the obstruction is. This is an invasive procedure that can increase the patient's risk of infection. If the malfunction cannot be isolated to a specific shunt component, replacement surgery must occur. An embodiment of the present invention may enable continuous monitoring of hydrocephalus shunt function. An embodiment of the invention may provide continuous monitoring through the product lifecycle, early warnings of malfunctions or clinical conditions, determinations of malfunction locations within the shunt system, and direct measurement of intracranial pressure from within the ventricular space.

There are current methods for noninvasive flow rate determination and blockage detection; however, the current methods lack the capacity to detect the precise location of a malfunction. In one embodiment, the device is implanted and therefore may have higher sensitivity and resolution than existing devices and may also be used to assess shunt function in the ventricular space. The present system may be able to interface with current shunt systems in the market, may be fully encapsulated, and may use both piezoresistive and electret sensor technology for pressure and flow sensing. Embodiments of the invention may enable faster treatment and better patient outcomes through monitoring and early detection of abnormal intracranial pressure, which may result in clinical events such as seizures, nausea and vomiting, loss of developmental progress, vision problems, poor coordination, loss of bladder control, and mental impairment [1].

### Magnitude of Hydrocephalus Shunt Malfunction

The global hydrocephalus shunt market in 2022 was $434 million, with a 4.4% compound annual growth rate (CAGR). By 2028, the global market for hydrocephalus shunts is projected to reach $562 million [2]. In the United States (US), the market in 2022 was $122 million, with a 3.5% CAGR, and is projected to reach $151 million by 2028 [3]. The per-patient cost for shunt-related primary procedures ranges from $137 to $814,748, with an average cost of $35,816. The cost burden of hydrocephalus shunt failures in the US was approximately $4 billion in 2022 and is projected to reach over $5 billion by 2029. In 2005, the cost of hospital admissions alone due to shunt failure exceeded $1 billion. Costs associated with the shunt implant procedure, complications due to infectious and noninfectious shunt failures, and replacement or removal procedures, include clinician reimbursement, hospital staff, inpatient hospital services, medicine, device reimbursement, material and utility costs, follow-on hospital visits, and business costs. Shunt placements and replacements each account for 46% of procedures, while shunt removals account for 8%.

Implementing the proposed product could result in a $60,000 cost saving for infectious and noninfectious shunt failures and would only represent an added cost of $500 at implant. Given the current number of patients with hydrocephalus, with 22,000 shunt failures occurring per year in the US and a cost saving of $60,000 per failure, implementing this product in every hydrocephalus shunt would save up to $1.32 billion per year in the US alone. At $500 per unit for 40,000 shunts implanted per year in the US, $1,000 per telemetry unit for 5,700 hospitals, and $1,000 per year for software, revenue would potentially reach $26 million per year. As the number of patients with hydrocephalus increases, revenue would increase proportionally.

### SUMMARY OF INVENTION

In one embodiment of the present invention, an acoustic sensor such as the Hearo electret sensor developed at Johns Hopkins University is used to determine the intracranial pressure (ICP) with high specificity and accuracy. Hearo is a flexible, electrostatic acoustic sensor that receives sound waves from a surface and converts them to a digital waveform. Current ICP measurement devices are implanted and removed in the clinic and cannot provide long-term tracking as they require the patient be in the intensive care unit for monitoring. Additionally, current ICP measurement devices do not directly measure the ICP. After implantation and during extended use, shunt systems may become obstructed at different locations within the system and mechanical malfunctions may occur, and current systems do not allow for the detection of malfunctions with any type of localization.

The inventive system allows for localization of malfunctions to specific shunt components. Detection and localization of malfunctions can significantly reduce the need for invasive surgery and allow clinicians to replace only the malfunctioning component. Replacing ventricular catheters requires open brain surgery; isolating malfunctions to the valve or distal catheter can preclude the need to preemptively replace the ventricular catheter.

An embodiment of the present device may be used to assess shunt function in the ventricular space, a capability that current solutions lack. In the event of a shunt malfunction, the patient may present with symptoms including headache, nausea, blindness, loss of motor function, speech difficulties, and cognitive impairment. The presence of symptoms often indicates a neurological emergency. Embodiments of the present invention enable early warnings to the patient and clinician of shunt malfunctions or clinical conditions that may need clinical intervention.

In some aspects, the techniques described herein relate to a system for monitoring intracranial pressure (ICP) of cerebrospinal fluid (CSF) and shunt flow in a hydrocephalus patient, the system including: a shunt configured to be deployed between a ventricular cavity and a peritoneal space of the patient, the shunt including: a ventricular catheter having a ventricular cavity end and a valve end, a peritoneal catheter having a valve end and distal end, a valve in fluid communication with the valve end of the ventricular catheter and the valve end of the peritoneal catheter, a first piezoelectric pressure sensor disposed on the ventricular catheter proximate to the valve end, a second piezoelectric pressure sensor disposed on the peritoneal catheter proximate to the valve end, a third piezoelectric pressure sensor disposed on the peritoneal catheter proximate to the distal end, and an intracranial electret pressure sensor disposed on the ventricular cavity end of the ventricular catheter; and a processing and transmission module including: a microcontroller, an analog-to-digital converter (ADC) in electrical communication with the microcontroller and the first, second, and third piezoelectric pressure sensors and the intracranial electret pressure sensor, and a wireless data transmitter in electrical communication with the microcontroller, wherein the microcontroller is configured to determine pressure values at each of said sensors based on data from the ADC, to compute shunt flow including ventricular catheter, peritoneal catheter, and valve flow rates based on said determined pressure values, and to cause the wireless data transmitter to transmit a reading including said pressure values and shunt flow to an external receiver.

In some aspects, the techniques described herein relate to a system, wherein the microcontroller includes a microprocessor and tangible computer readable media storing instructions that cause the microprocessor to effect the pressure values determinations, the shunt flow computation, and the wireless data transmission. In some aspects, the techniques described herein relate to a system, wherein the processing and transmission module further includes a power reception coil electrically coupled to provide power to the microcontroller, wireless data transmitter, and ADC. In some aspects, the techniques described herein relate to a system, further including an inductive power/data link module, said module configured to inductively provide power to the power reception coil, receive transmissions of the reading from the wireless data transmitter, and transmit said reading to a clinical telemetry unit.

In some aspects, the techniques described herein relate to a system, further including the clinical telemetry unit, said unit including a receiver configured to receive said reading, a processor, and a display, said processor configured to cause the display to display an ICP value of said patient and shunt flow based on said reading. In some aspects, the techniques described herein relate to a system, wherein the processor is further configured to cause the display to display an alert of a determined malfunction, an out-of-threshold shunt flow, or an out-of-threshold ICP based on said reading. In some aspects, the techniques described herein relate to a system, wherein the processor is further configured to cause the display to display a shunt malfunction location based on said reading. In some aspects, the techniques described herein relate to a system, wherein the clinical telemetry unit further includes storage and the processor is further configured to cause a plurality of said readings over time to be stored in the storage, and the processor is further configured to cause the display to display a data aggregation including averaged ICP or an ICP waveform based on said plurality of said readings.

In some aspects, the techniques described herein relate to a system wherein the intracranial electret pressure sensor: is planar in conformation, a sensing surface thereof faces the CSF, and an acoustic impedance thereof is tuned to match an acoustic impedance of the CSF. In some aspects, the techniques described herein relate to a system wherein the first, second, and third piezoelectric pressure sensors are composed of a porous medium containing evenly distributed carbon nanotubes. In some aspects, the techniques described herein relate to a system wherein the intracranial electret pressure sensor: is circular in conformation, a sensing surface thereof faces the ventricular catheter, and an acoustic impedance thereof is tuned to match an acoustic impedance of the ventricular catheter. In some aspects, the techniques described herein relate to a system wherein a conformation of the intracranial electret pressure sensor is one of circular or planar. In some aspects, the techniques described herein relate to a system wherein the intracranial electret pressure sensor is tuned to match an acoustic impedance of the CSF.

In some aspects, the techniques described herein relate to a method for monitoring intracranial pressure (ICP) of cerebrospinal fluid (CSF) and shunt flow in a hydrocephalus patient, the method including: deploying a shunt between a ventricular cavity and a peritoneal space of the patient, the shunt including: a ventricular catheter having a ventricular cavity end and a valve end, a peritoneal catheter having a valve end and distal end, a valve in fluid communication with the valve end of the ventricular catheter and the valve end of the peritoneal catheter, a first piezoelectric pressure sensor disposed on the ventricular catheter proximate to the valve end, a second piezoelectric pressure sensor disposed on the peritoneal catheter proximate to the valve end, a third piezoelectric pressure sensor disposed on the peritoneal catheter proximate to the distal end, and an intracranial electret pressure sensor disposed on the ventricular cavity end of the ventricular catheter; determining pressure values at each of said sensors based electrical values therefrom, said pressure values including ICP; and computing shunt flow including ventricular catheter, peritoneal catheter, and valve flow rates based on said determined pressure values.

In some aspects, the techniques described herein relate to a method, further including a step of determining a shunt malfunction based on said computed shunt flow. In some aspects, the techniques described herein relate to a method, further including a step of determining a shunt malfunction location based on said computed shunt flow. In some aspects, the techniques described herein relate to a method, further including providing an alert to a device of the patient or a device of a clinician of the patient. In some aspects, the techniques described herein relate to a method, further including providing a waveform of a plurality of ICP values over time.

In some aspects, the techniques described herein relate to a method wherein the intracranial electret pressure sensor is tuned to match an acoustic impedance of the CSF. In some aspects, the techniques described herein relate to a method wherein a conformation of the intracranial electret pressure sensor is one of circular or planar. In some aspects, the techniques described herein relate to a method wherein the intracranial electret pressure sensor: is circular in conformation, a sensing surface thereof faces the ventricular catheter, and an acoustic impedance thereof is tuned to match an acoustic impedance of the ventricular catheter. In some aspects, the techniques described herein relate to a method wherein the first, second, and third piezoelectric pressure sensors are composed of a porous medium containing evenly distributed carbon nanotubes. In some aspects, the techniques described herein relate to a method wherein the intracranial electret pressure sensor: is planar in conformation, a sensing surface thereof faces the CSF, and an acoustic impedance thereof is tuned to match an acoustic impedance of the CSF.

### BRIEF DESCRIPTION OF THE DRAWINGS

The accompanying drawings, which are included to provide a further understanding of the disclosed subject matter, are incorporated in, and constitute a part of this specification. The drawings illustrate exemplary embodiments of the disclosed invention and together with the detailed description, serve to demonstrate the fundamental principles of said invention.

FIG. 1 illustrates an exemplary hydrocephalus shunt monitoring system according to an embodiment of the invention.

FIG. 2 illustrates an exemplary sensor layout according to embodiments of the invention.

FIG. 3 is an exemplary schematic of wireless power and data transfer inductive links in accordance with embodiments of the invention.

FIG. 4 illustrates an exemplary process according to embodiments of the invention.

FIG. 5 illustrates an exemplary clinical telemetry unit/app.

### DETAILED DESCRIPTION

Reference will now be made in detail to exemplary embodiments, discussed with regard to the accompanying drawings. In some instances, the same reference numbers will be used throughout the drawings and the following description to refer to the same or like parts. Unless otherwise defined, technical and/or scientific terms have the meaning commonly understood by one of ordinary skill in the art. The disclosed embodiments are described in sufficient detail to enable those skilled in the art to practice the disclosed embodiments. It is to be understood that other embodiments may be utilized and that changes may be made without departing from the scope of the disclosed embodiments. For example, unless otherwise indicated, method steps disclosed in the figures can be rearranged, combined, or divided without departing from the envisioned embodiments. Similarly, additional steps may be added or steps may be removed without departing from the envisioned embodiments. Thus, the materials, methods, and examples are illustrative only and are not intended to be necessarily limiting.

### Overview

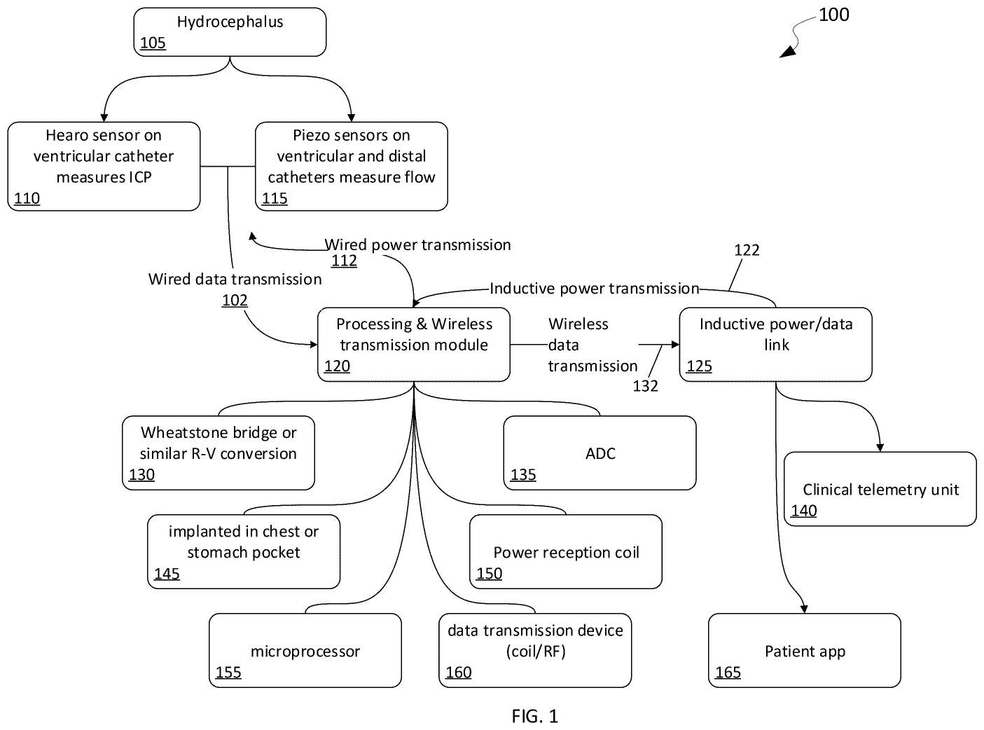

With reference to embodiments 100 illustrated in FIG. 1, systems and methods for monitoring CSF in hydrocephalus 105 and shunt function are provided. ICP may be measured 100 and piezo sensor may measure catheter flow 115. Information may be transmitted by wire 102 to a processing and wireless transmission module 120 and power may be provided by wire 112. Module 120 may have elements for R-V conversion 130, analog-to-digital conversion 135, a power reception coil 150, a data transmission device 160, and microprocessor 155. Module 120 may be implanted in a patient chest or stomach pocket 145. Module 120 may receive power inductively 122 and transmit data wirelessly 132 to an inductive power/data link 125. Link 125 may provide data to a clinical telemetry unit 140 and or to a patient app 165.

An embodiment of the present invention operates on the principle that pressure differentials at both ends of a tube correlate to the flow rate of fluid inside the tube. The proximal and distal catheters may be instrumented with one sensor at either end. Three of the sensors may be piezoelectric polymer sensors, such as those developed at Johns Hopkins University [4], and the fourth may be an electret sensor. The pressure differential between the first two sensors may correlate to the flow rate in the proximal catheter; the pressure differential between the second and third sensors may correlate to the flow rate in the valve, and the pressure differential between the third and fourth sensors may correlate to the flow rate in the distal catheter. Pressure differential readings may be obtained, amplified by an integrated circuit, and digitally processed to determine average values over time. The device may also enable continuous monitoring of intracranial pressure (ICP) 110 using an electret sensor which may be mounted on the ventricular catheter. The electret sensor may be the ‘Hearo’ device developed at Johns Hopkins University, a flexible, electrostatic acoustic sensor that receives sound waves from a surface and converts them to a digital waveform [5]. Hearo features a single electret diaphragm layer that may be tuned to specifically match the acoustic impedances of multiple materials [6]. A signal processing and wireless power and data transmission module 120 is integrated into the system. A detection algorithm triggers an alert to notify the patient and send data to the clinician of a malfunction, sub-optimal shunt operation, or onset of a clinical condition. In the clinic, the pressure differential readings are transmitted wirelessly and displayed in real-time to locate the specific segment of the shunt that is malfunctioning. The clinician can also display readings from the lifetime of the shunt to monitor changes in shunt function and ICP over time.

The electret sensor may be used to sense ICP and may be instrumented on the end of the ventricular catheter, with the sensing surface facing the (CSF). The acoustic impedance of the sensing surface may be tuned to match that of CSF. Piezoresistive sensors may be constructed using piezoresistive. The acoustic impedance of the polymer may be tuned to match that of the silicone comprising the catheter body. The adhesion of the sensors to the catheter surface may present a challenge, as any flexion or compression of the catheter due to a change in CSF where the flow must be accurately captured by the sensors. Silicone adhesive may be used to fix the sensors to the catheters. Further, the sensors must be constructed suitably for long-term use, and as such the sensors may be encapsulated with biocompatible material for implant. Long-term operation of the sensors may require maintenance or calibration to account for physical degradation or signal drift.

In one embodiment there is a wireless power and data transmission module 120, such as that designed and fabricated based on a miniaturized inductively powered neural stimulator chip developed by Dr. Ralph Etienne Cummings from Johns Hopkins University [7]. The module 120 may have an implanted receiver constructed using analog electrical components designed to sit in a chest or abdominal skin pocket, similar to commercial deep brain stimulation device. A transmitter 125 may be constructed and may sit on the patient's skin, over the receiver site. The module 120 may incorporate passive analog electrical components and may be powered using a long-lasting rechargeable battery. Technical challenges and risks in this section include attenuation of the wireless signal through the skull and safe operation of the implanted system, and low-power operation of the module over the life of the implant. In some embodiments, the receiver is encapsulated for biocompatibility. In some embodiments, the telemetry module is compatible with the wireless module of the integrated circuit. Strategies to minimize noise due to external movement and remove confounding data, and to compensate for sensor drift may be implemented with analog and digital filtering and other forms of digital signal processing. In some embodiments, wireless system 300 as illustrated in FIG. 3 can effect elements of transmitter 125 and module 120.

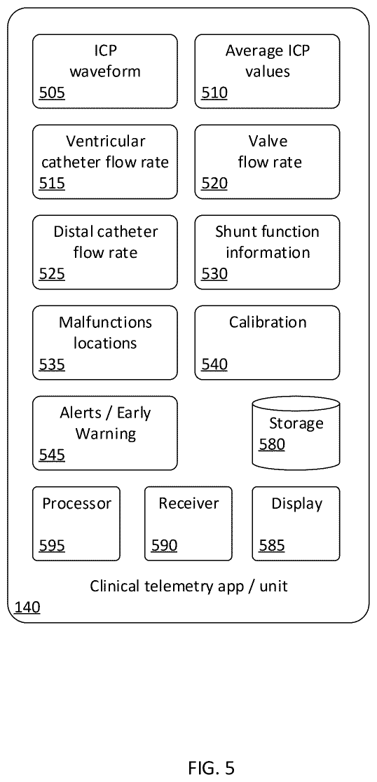

With reference to FIG. 5, an easy-to-use application/unit 140 provides for clinicians to display the ICP waveform 505, average ICP values 510, and flow rates within the ventricular catheter 515, valve 520, and distal catheter 525, and to display information about the shunt's function 530 and location of malfunctions 535. A calibration method may be defined and implemented 540 to ensure the system is calibrated properly for each individual patient. This calibration method may account for the behavior of the sensors and varying physiological conditions that affect flow rate. Digital signal processing algorithms may account for confounding factors such as patients being in a prone versus standing position, sleeping versus awake, variation in activity levels, stress, and concurrent clinical conditions. Algorithms may analyze time series data, utilize clinically validated indicators of confounding factors' impact on CSF pulsatile waveforms, automatically detect confounding factors, and filter outlier data and noise. Calibration may be hardcoded into the system and adapted to individual patients autonomously or done via software in the clinic. The system may also be programmable by the clinician to adjust parameters including sensitivity and sampling rate. An algorithm may provide for malfunction detection and localization based on the measured flow rate. An early warning mechanism 545 may be built into the system to alert the patient and clinician of a malfunction or of a change in ICP before the onset of symptoms, based on the measured flow rates and ICP. The warning may be automatically transmitted to the patient's device of choice and to the clinician. The architecture and design of this alert system may require patient and clinician input. The clinician may be able to adjust the parameters triggering an alert.

Application/unit 140 may comprise a processor 595, a data receiver 590 to receive data from inductive power/data link module 125, a display 585, and storage 580.

One embodiment of the device design uses wired connections between the sensors and the power and data transmission module. Long-term use has the potential to lead to wire breakage. In other embodiments, wireless communication between the sensors and inductive link may be provided.

### A Validation System

A benchtop testing system to validate the use of sensors to display pressure readings due to fluid flow within flexible tubing includes a pump to continuously drive water through the tubing and a valve may be used to control the flow rate. The tubing may be secured on a vibration-isolated benchtop to prevent interference due to external noise or vibrations. The resistive sensors may act as one leg of a quarter-bridge Wheatstone bridge. A variable resistor may be used to balance the Wheatstone bridge. An instrumentation amplifier may use three LM741 operational amplifiers. An Arduino Uno may be used to provide 5V power and may be connected to a Mac laptop to display the signal from the force sensors in real-time. Reducing the flow rate corresponded to a reduction in voltage output.

Piezoresistive sensors may be fabricated using polymer and copper tape and may be connected to the Wheatstone bridge. One bridge resistor may be varied to match the polymer's baseline resistance and sensor readings were taken using an Arduino Uno and the Arduino IDE by lightly squeezing the sensor. Applied force corresponded to a decrease in resistance and an increase in signal output. The ventricular catheter instrumented with sensors was connected to a water-filled syringe and a flow test was conducted. The sensors were reported in the literature [4] to detect pressures as low as 1 Pa; the minimum ICP was taken to be 400 Pa.

The Hearo sensor was connected to a laptop computer through an audio preamplifier with phantom power, and the output was visualized as an audio waveform in Logic Pro X. A flow test was conducted on the benchtop continuous flow model by placing the Hearo sensor on top of the flexible tubing, varying the flow rate, and measuring the output. The waveform clearly displayed a reduced amplitude when the flow was decreased. A second test was conducted to measure Hearo's response to water pressure fluctuations in a sealed chamber. The sensor was reported in the literature [6] to have a sensitivity of 2V/N.

### Design Theory

Aspects of the present invention are based on the principle that pressure differentials at both ends of a tube correlate to the flow rate of fluid inside the tube. The Hagen-Poiseuille equation [8] describes the laminar flow of a Newtonian, incompressible fluid in a long, narrow pipe. Rewriting the equation to give flow rate yields Eq. 1, where Δp is the pressure difference between the two ends, R is the pipe radius, μ is the dynamic viscosity, L is the length of the pipe, and Q is the volumetric flow rate.

$$Q = \frac{Δ p π {R}^{4}}{8 μ L} . \qquad ( Eq . 1 )$$

CSF flow rates were calculated using a range of 42 different intracranial pressures [9] and an average of CSF viscosity values obtained from the literature in a Desu ventricular catheter with an open distal end, for different internal blockage conditions from free flow to a complete obstruction. A peritoneal pressure of 3 mmHg (400 Pa) was taken as an average value from the literature [11]. An obstruction was modeled as a decrease in pipe radius. The flow rates obtained match those reported in the literature. At 80% occlusion, the flow rate drops to 0 ml/min.

### In Use

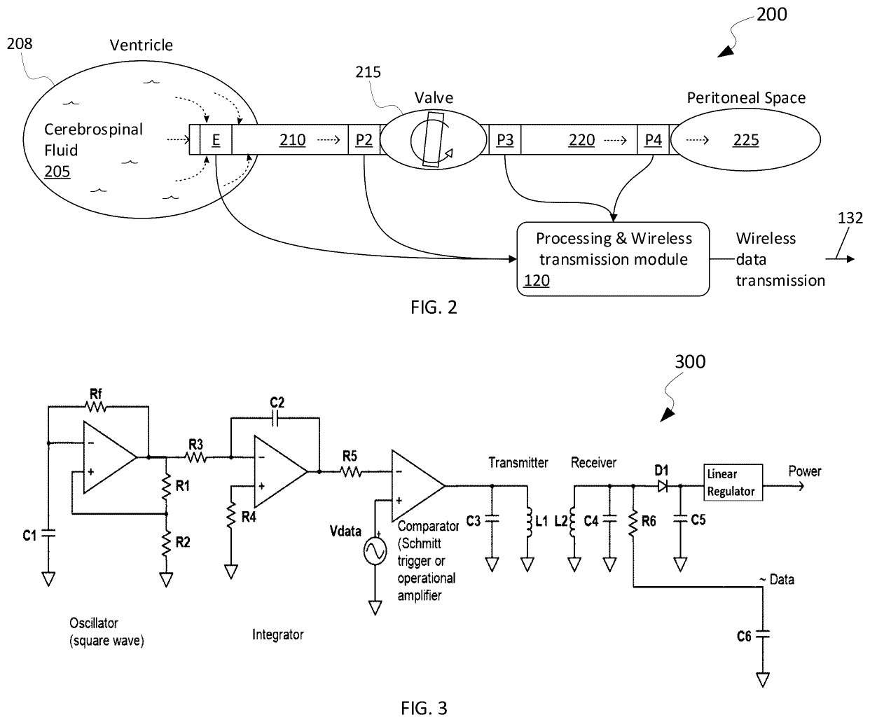

With reference to FIG. 2, in accordance with one embodiment of system 200 there may be three piezoelectric pressure sensors P2, P3, and P4 affixed to ends of ventricular catheter 210 and peritoneal catheter 220, to sense pressure applied by CSF 205 within the catheters, and one intracranial pressure sensor E on the surface of the ventricular catheter 210. Each pressure sensor may acquire continuous pressure values from the catheter external surface due to the CSF flow. Pressure differentials between each sensor pair may correspond to a flow rate measurement for that segment. A calibration curve of pressure values for known flow rates together with acquired pressure differential readings may map back to the calibrated flow rates to obtain flow rates in vivo. Sensors E and P2 may provide the flow rate for proximal catheter 210, sensors P2 and P3 may provide the flow rate for valve 215, and sensors P3 and P4 may provide the flow rate for distal catheter 220 into peritoneal space 225. Electret sensor E may be mounted on ventricular catheter 210 to continuously monitor intracranial pressure within ventricle 208.

### Detailed Description of the Sensors

Sensor E may be a modification of the Hearo sensor developed by Dr. James West at Johns Hopkins University [5,6]. The sensor may feature a single electret diaphragm layer that may be tuned to specifically match the acoustic impedances of multiple materials. The sensor captures acoustic energy more efficiently than sensors used in current acoustic transducers. The size and shape of the sensor's microstructures may be tuned to affect the frequency response. The sensor may be repurposed to measure changes in pressure. The sensor may be miniaturized and may be used in both a planar and in a circular conformation. The planar conformation may be used to sense intracranial pressure and may be instrumented on the end of the ventricular catheter, with the sensing surface facing the cerebrospinal fluid. The acoustic impedance of the sensing surface may be tuned to match that of CSF. The circular conformation may have the sensing surface facing the catheter body and may sense pressure changes from within the catheter. The acoustic impedance of the sensing surface may be tuned to match that of the silicone comprising the catheter body. The acoustic impedance of the current sensor iteration may be tuned to 1-2.5 MRayls, covering skin, fresh and salt water, and most plastics. The sensor may be capsulated in biocompatible material suitable for long-term use in humans.

Ultrasensitive piezoresistive sensors [4] include those which were developed by Dr. Sung Hoon Kang at Johns Hopkins University. These sensors may utilize a novel polymer material composed of a porous medium containing evenly distributed carbon nanotubes. The sensors may be greater than 50 times more sensitive than traditional piezoresistive sensors; which may be enough to detect the pressure changes due to varying CSF flow in shunt catheters. Sensors may be constructed using this piezoresistive polymer and tested for optimal polymer thickness and sensor construction modality. The acoustic impedance of the polymer may be measured and tuned to match that of silicone. The adhesion of the sensors to the catheter surface may present a challenge, as any flexion or compression of the catheter due to a change in CSF flow must be accurately captured by the sensors. An adhesive may not provide a slipping surface between the catheter and the sensor body and may not be stiffer than the catheter itself; energy or signal loss through the adhesive may be minimized. Silicone adhesive may be used to fix the sensors to the catheters; the polymer composite may be based on silicone, and the surface of the shunt catheters may be silicone. Further, the sensors may be constructed suitably for long-term use. Sensors may be encapsulated with biocompatible material for implant.

### Detailed Description of the Wireless Power and Data Transmission Module

With reference to FIG. 3, the wireless power and data transmission system 300 may have an implanted receiver constructed using analog electrical components designed to sit in a chest or abdominal skin pocket, similar to commercial deep brain stimulation device. The receiver may include an inductor and a linear regulator. A transmitter may be constructed using analog electrical components and may sit on the patient's skin, over the receiver site. A square wave oscillator, integrator, comparator (Schmitt trigger or operational amplifier) and inductor may be used. Both the receiver and transmitter use passive electrical components such as resistors, capacitors, operational amplifiers, inductors, and diodes. A battery is incorporated into the module. The receiver may obtain data from the sensors and may wirelessly transmit the data to the transmitter, which may store the data and may transmit it to clinician and patient modules. The transmitter may be battery-powered using a long-lasting rechargeable battery and may wirelessly transmit power to the receiver. This design may allow for minimizing the footprint of the implanted portion of the system.

### Detailed Description of Clinician Application Design

A simple application may display the ICP waveform, average ICP values, and flow rate within the ventricular catheter, valve, and distal catheter. The application may run on a laptop or desktop computer and may present both real-time and stored data from the device. To ensure the system is calibrated properly for each individual patient, a calibration method may be defined and implemented. This may account for the behavior of the sensors and varying physiological conditions that affect flow rate (prone vs. standing, sleeping vs. awake, activity levels, patient etiologies).

The clinician can use telemetry in the clinic to obtain flow rate output vs. expected output for each segment of the shunt system, providing them with information about the shunt system's function. The clinician may use stored data to display changes in the shunt system's function from the implant. A software module may display the shunt data in an intuitive format. The system may be programmable by the clinician to adjust parameters including sensitivity and sampling rate. An algorithm may detect and localize malfunction based on the measured flow rate. Algorithm development may involve analyzing time series data, utilizing clinically validated indicators of confounding factors' impact on CSF pulsatile waveforms, automatic detection of confounding factors, and filtering of outlier data and noise. The telemetry module may be compatible with the wireless module of the integrated circuit.

An early warning mechanism may be built into the system to alert the patient and clinician of a malfunction or of a change in ICP before the onset of symptoms, based on the measured flow rates and ICP. The warning will be automatically transmitted to the patient's device of choice and to the clinician. The clinician may be able to adjust the parameters that trigger an alert.

### PROCESS

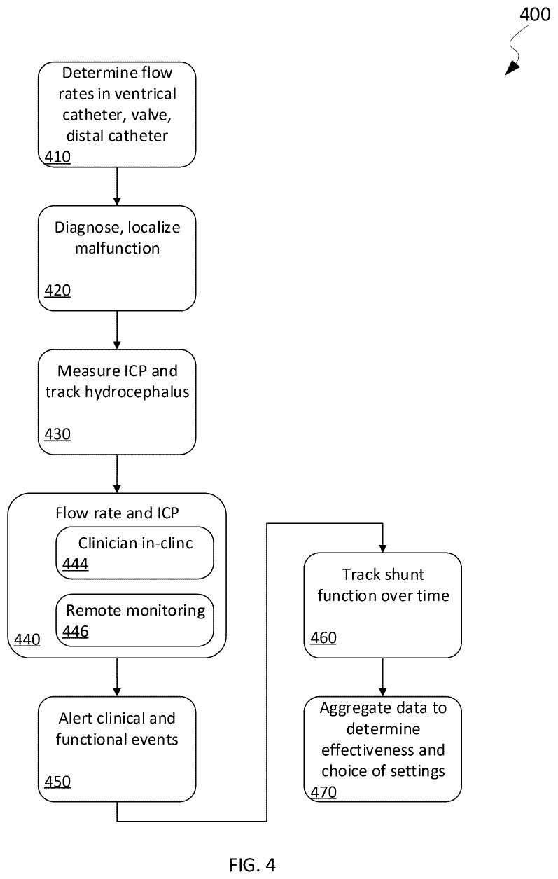

With reference to FIG. 4, there is a process 400 in accordance with the present invention having several steps.

In step 410, Pressure differentials between sensors may be used to determine the flow rate of cerebrospinal fluid in the ventricular catheter, valve, and distal catheter of hydrocephalus shunt systems.

In step 420, The measured flow rate in the shunt system may be used to diagnose malfunctions and to localize malfunctions to the ventricular catheter, valve, and distal catheter.

In step 430, The measured intracranial pressure (ICP) may be used to track the condition of the patient's hydrocephalus.

In step 440, The flow rate and ICP may be monitored by a clinician using an implanted system.

In step 444, the flow rate and ICP can be acquired by the clinician in the clinic using telemetry.

In step 446, the system can send flow rate and ICP data to the clinician remotely at user-defined intervals.

In step 450, An alert system can provide an early warning to both the patient and the clinician of clinical events and shunt malfunctions.

In step 460, Recorded flow rate and ICP data may be used to track the function of the shunt over time.

In step 470, Flow rate and ICP data can be aggregated and studied to support shunt effectiveness and choice of shunt settings. In one embodiment, device integration with hydrocephalus shunt systems enables continuous monitoring of shunt function, early warning of malfunctions, and allows clinicians to locate the malfunction site.

### REFERENCES

[1] National Institute of Neurological Disorders and Stroke. “Hydrocephalus.” Downloaded May 4, 2025 from www.ninds.nih.gov/health-information/disorders/hydrocephalus; copy in patent file, hereby incorporated by reference.
[2] Hydrocephalus Shunts Market: Global Industry Trends, Share, Size, Growth, Opportunity and Forecast 2023-2028. (2023). IMARC Group. Viewed at www.researchandmarkets.com/reports/5820670/hydrocephalus-shunts-market-global-industry.
[3] North America Hydrocephalus Shunts Market Forecast to 2028-COVID-19 Impact and Regional Analysis—by Age Group (Pediatric and Adults), Type (Ventriculo-Peritoneal, Ventriculo-Atrial, Ventriculo-Pleural, and Lumbo-Peritoneal), Product (Hydrocephalus Valves, Hydrocephalus Catheters, Neuronavigation Systems, and Others), and End User (Hospitals, Ambulatory Surgical Centers, and Others). (2023). The Insight Partners. Viewed at www.businessmarketinsights.com/reports/north-america-hydrocephalus-shunts-market/.
[4] Li, Jing, et al. “Ultrasensitive, flexible, and low-cost nanoporous piezoresistive composites for tactile pressure sensing.” Nanoscale 11.6 (2019): 2779-2786.
[5] West et al. “Impedance-matched acoustic transducer.” Int'l patent application publication no. WO2022076770A1, April 14, 2022.
[6] Rennoll, Valerie, et al. “Evaluating the impact of acoustic impedance matching on the airborne noise rejection and sensitivity of an electrostatic transducer.” The Journal of the Acoustical Society of America 149.4_Supplement (2021): A23-A23.
[7] Khalifa, Adam, et al. “A compact, low-power, fully analog implantable microstimulator.” 2016 IEEE International Symposium on Circuits and Systems (ISCAS). IEEE, 2016. 2435-2438.
[8] Ostadfar, Ali. Biofluid Mechanics: Principles and Applications. Netherlands, Elsevier Science, 2016.
[9] Norager, Nicolas Hernandez, et al. “Reference values for intracranial pressure and lumbar cerebrospinal fluid pressure: a systematic review.” Fluids and Barriers of the CNS 18 (2021): 1-10.
[10] Bloomfield, I. G., I. H. Johnston, and L. E. Bilston. “Effects of proteins, blood cells and glucose on the viscosity of cerebrospinal fluid.” Pediatric neurosurgery 28.5 (1998): 246-251.
[11] Depauw, Paul RAM, et al. “The significance of intra-abdominal pressure in neurosurgery and neurological diseases: a narrative review and a conceptual proposal.” Acta neurochirurgica 161.5 (2019): 855-864.

1. A system for monitoring intracranial pressure (ICP) of cerebrospinal fluid (CSF) and shunt flow in a hydrocephalus patient, the system comprising:
a shunt configured to be deployed between a ventricular cavity and a peritoneal space of the patient, the shunt including:
a ventricular catheter having a ventricular cavity end and a valve end,
a peritoneal catheter having a valve end and distal end,
a valve in fluid communication with the valve end of the ventricular catheter and the valve end of the peritoneal catheter,
a first piezoelectric pressure sensor disposed on the ventricular catheter proximate to the valve end,
a second piezoelectric pressure sensor disposed on the peritoneal catheter proximate to the valve end,
a third piezoelectric pressure sensor disposed on the peritoneal catheter proximate to the distal end, and
an intracranial electret pressure sensor disposed on the ventricular cavity end of the ventricular catheter; and
a processing and transmission module including:
a microcontroller,
an analog-to-digital converter (ADC) in electrical communication with the microcontroller and the first, second, and third piezoelectric pressure sensors and the intracranial electret pressure sensor, and
a wireless data transmitter in electrical communication with the microcontroller,
wherein the microcontroller is configured to determine pressure values at each of said sensors based on data from the ADC, to compute shunt flow comprising ventricular catheter, peritoneal catheter, and valve flow rates based on said determined pressure values, and to cause the wireless data transmitter to transmit a reading comprising said pressure values and shunt flow to an external receiver.

2. The system according to claim 1, wherein the microcontroller comprises a microprocessor and tangible computer readable media storing instructions that cause the microprocessor to effect the pressure values determinations, the shunt flow computation, and the wireless data transmission.

3. The system according to claim 1, wherein the processing and transmission module further comprises a power reception coil electrically coupled to provide power to the microcontroller, wireless data transmitter, and ADC.

4. The system according to claim 3, further comprising an inductive power/data link module, said module configured to inductively provide power to the power reception coil, receive transmissions of the reading from the wireless data transmitter, and transmit said reading to a clinical telemetry unit.

5. The system according to claim 4, further comprising the clinical telemetry unit, said unit comprising a receiver configured to receive said reading, a processor, and a display, said processor configured to cause the display to display an ICP value of said patient and shunt flow based on said reading.

6. The system according to claim 5, wherein the processor is further configured to cause the display to display an alert of a determined malfunction, an out-of-threshold shunt flow, or an out-of-threshold ICP based on said reading.

7. The system according to claim 5, wherein the processor is further configured to cause the display to display a shunt malfunction location based on said reading.

8. The system according to claim 5, wherein the clinical telemetry unit further comprises storage and the processor is further configured to cause a plurality of said readings over time to be stored in the storage, and the processor is further configured to cause the display to display a data aggregation comprising averaged ICP or an ICP waveform based on said plurality of said readings.

9. The system of claim 1 wherein the intracranial electret pressure sensor:
is planar in conformation,
a sensing surface thereof faces the CSF, and
an acoustic impedance thereof is tuned to match an acoustic impedance of the CSF.

10. The system of claim 1 wherein the first, second, and third piezoelectric pressure sensors are composed of a porous medium containing evenly distributed carbon nanotubes.

11. The system of claim 1 wherein the intracranial electret pressure sensor:
is circular in conformation,
a sensing surface thereof faces the ventricular catheter, and
an acoustic impedance thereof is tuned to match an acoustic impedance of the ventricular catheter.

12. (canceled)

13. The system of claim 1 wherein the intracranial electret pressure sensor is tuned to match an acoustic impedance of the CSF.

14. A method for monitoring intracranial pressure (ICP) of cerebrospinal fluid (CSF) and shunt flow in a hydrocephalus patient, the method comprising:
deploying a shunt between a ventricular cavity and a peritoneal space of the patient, the shunt including:
a ventricular catheter having a ventricular cavity end and a valve end,
a peritoneal catheter having a valve end and distal end,
a valve in fluid communication with the valve end of the ventricular catheter and the valve end of the peritoneal catheter,
a first piezoelectric pressure sensor disposed on the ventricular catheter proximate to the valve end,
a second piezoelectric pressure sensor disposed on the peritoneal catheter proximate to the valve end,
a third piezoelectric pressure sensor disposed on the peritoneal catheter proximate to the distal end, and
an intracranial electret pressure sensor disposed on the ventricular cavity end of the ventricular catheter;
determining pressure values at each of said sensors based electrical values therefrom, said pressure values including ICP; and
computing shunt flow comprising ventricular catheter, peritoneal catheter, and valve flow rates based on said determined pressure values.

15. The method of claim 14, further comprising a step of determining a shunt malfunction based on said computed shunt flow.

16. (canceled)

17. The method of claim 15, further comprising providing an alert to a device of the patient or a device of a clinician of the patient.

18. The method of claim 14, further comprising providing a waveform of a plurality of ICP values over time.

19. The method of claim 14 wherein the intracranial electret pressure sensor is tuned to match an acoustic impedance of the CSF.

20. (canceled)

21. The method of claim 14 wherein the intracranial electret pressure sensor:
is circular in conformation,
a sensing surface thereof faces the ventricular catheter, and
an acoustic impedance thereof is tuned to match an acoustic impedance of the ventricular catheter.

22. The method of claim 14 wherein the first, second, and third piezoelectric pressure sensors are composed of a porous medium containing evenly distributed carbon nanotubes.

23. The method of claim 14 wherein the intracranial electret pressure sensor:
is planar in conformation,
a sensing surface thereof faces the CSF, and
an acoustic impedance thereof is tuned to match an acoustic impedance of the CSF.
